(19) United States
(12) Patent Application Publication
(10) Pub. No.: US 2024/0373134 A1
(43) Pub. Date: Nov. 7, 2024

(54) SYSTEMS AND METHODS FOR MEDICAL IMAGE COLOR CALIBRATION AND CORRECTION

(71) Applicant: Boston Scientific Scimed, Inc., Maple Grove, MN (US)

(72) Inventors: Kirsten VIERING, Newton, MA (US); Louis J BARBATO, Franklin, MA (US); Jeffrey Frank HATALSKY, Framingham, MA (US)

(73) Assignee: Boston Scientific Scimed, Inc., Maple Grove, MN (US)

(21) Appl. No.: 18/646,864

(22) Filed: Apr. 26, 2024

Related U.S. Application Data
(60) Provisional application No. 63/499,824, filed on May 3, 2023.

(51) Int. Cl. H04N 23/76; G06T 7/80; G06T 7/90; H04N 23/84

(52) U.S. Cl. CPC H04N 23/76; G06T 7/80; G06T 7/90; H04N 23/84; G06T 2207/10024; G06T 2207/30004

(57) ABSTRACT

Systems and methods for color correcting images are described. An example method includes receiving, at a first and second time: first and second image data from an imaging device of a medical imaging system captured during a medical procedure; and a first and second value of an operating parameter of a light source of the medical imaging system, where a color of light emitted from the light source shifts based on values of the operating parameter. A first color shift is determined based on the first value, and a color correction function is applied to the first image data to compensate for the first color shift to generate first color corrected image data. A second color shift is determined based on the second value, and the color correction function is applied to the second image data to compensate for the second color shift to generate second color corrected image data.

### CROSS-REFERENCE TO RELATED APPLICATIONS

This application claims the benefit of priority from U.S. Provisional Application No. 63/499,824, filed May 3, 2023, which is incorporated by reference herein in its entirety.

### TECHNICAL FIELD

The disclosure relates generally to medical imaging systems and related methods of use. More specifically, aspects of the disclosure pertain to color calibration and correction processes for endoscopic imaging systems.

### BACKGROUND

Endoscopic imaging systems configured to capture images of a target site during a medical procedure include, among other devices, at least one light source and at least one imaging device. Some endoscopic imaging systems may drive the light source at a constant current throughout the medical procedure, and the imaging device gain and exposure time may be adjusted to achieve acceptable pixel saturation. To improve lighting control, other endoscopic imaging systems may adjust a light intensity of the light source first, and only adjust the imaging device gain and exposure time once the dynamic range of the light source has been exhausted. However, adjustment of the light intensity changes an amount of current flowing through the light source, and as the current changes, a spectrum of the light emitted by the light source varies. Variations in other operating parameters of the light source, such as a temperature, can also cause variation in the spectrum of the light emitted by the light source. The variation in the spectrum results in a color shift affecting how features of the target site are captured and reproduced by the endoscopic imaging system.

### SUMMARY

An example method for color correcting images performed by a computing system may include receiving, at a first time: first image data from an imaging device of a medical imaging system captured during a medical procedure; and a first value of an operating parameter of a light source of the medical imaging system, where a color of light emitted from the light source may shift based on values of the operating parameter. The example method may also include determining a first color shift based on the first value, applying a color correction function to the first image data to compensate for the first color shift to generate first color corrected image data, and providing, to a display associated with the computing system, the first color corrected image data for display. The example method may further include receiving, at a second time different from the first time: second image data from the imaging device captured during the medical procedure; and a second value of the operating parameter of the light source, where the second value may be different from the first value. The method may yet further include determining a second color shift based on the second value, applying the color correction function to the second image data to compensate for the second color shift to generate second color corrected image data, and providing, to the display, the second color corrected image data for display.

In any of the exemplary methods disclosed herein, a plurality of color shifts in light emitted by one or more light sources of one or more medical imaging systems, including at least the light source of the medical imaging system may be characterized across a plurality of values of the operating parameter to obtain and store color shift data. To determine the first color shift, first color shift data corresponding to the first value within the stored color shift data may be identified. The first color shift data may indicate a measured change in pixel intensity values of one or more image data components based on the first color shift of the light emitted by the light source when the operating parameter has the first value. To apply the color correction function to the first image data, one or more first compensation coefficients of the color correction function may be determined based on the first color shift data to compensate for the measured change in the pixel intensity values of the one or more image data components. The first color shift data may further indicate a timing associated with the measured change in the pixel intensity values of one or more image data components when the operating parameter has the first value, and the color correction function may be applied to the first image data based on the timing.

In additional aspects, to determine the second color shift, second color shift data corresponding to the second value within the stored color shift data may be identified. The second color shift data may indicate a measured change in pixel intensity values of one or more image data components based on the second color shift of the light emitted by the light source when the operating parameter has the second value. To apply the color correction function to the second image data, one or more second compensation coefficients of the color correction function may be determined based on the second color shift data to compensate for the measured change in the pixel intensity values of the one or more image data components. The second color shift data may further indicate a timing associated with the measured change in the pixel intensity values of one or more image data components when the operating parameter has the second value, and the color correction function may be applied to the second image data based on the timing.

In some aspects, a difference between the second value and the first value may be determined to be above a predefined threshold value. The operating parameter may be a current of the light source, and a first value of the current and a second value of the current may be received from the light source at the first time and the second time, respectively. The operating parameter may be a temperature of the light source, and a first value of the temperature and a second value of the temperature may be received from a temperature sensor positioned adjacent to the light source at the first time and the second time, respectively. The operating parameter may be a temperature of the light source, and a first value of the temperature and a second value of the temperature may be inferred based on a known ambient temperature, a known junction thermal resistance of the light source, and a value of a current of the light source received at the first time and the second time, respectively.

In other aspects, a first timing associated with the first color shift may be determined. The first timing may include a measured time period from a detection of the operating parameter of the light source operating at the first value to an observation of the first color shift. The color correction function may be applied to the first image data based on the first timing. A second timing associated with the second color shift may be determined. The second timing may include a measured time period from a detection of the operating parameter of the light source operating at the second value to an observation of the second color shift. The color correction function may be applied to the second image data based on the second timing. The operating parameter may be a first operating parameter, and, at the first time, a first value of a second operating parameter of the light source may be received in addition to the first value of the first operating parameter. The first operating parameter at the first value may be determined as having a more dominant effect on the color of the light emitted from the light source than the second operating parameter at the first value, where the determination may cause the first color shift and an associated timing of the first color shift to be determined based on the first value of the first operating parameter.

A computing system for color correcting images that is communicatively connectable to a medical imaging system may include a data store, at least one memory, and one or more processors including an image processor. The data store may store color shift data obtained from a characterization of a plurality of color shifts in light emitted by one or more light sources of one or more medical imaging systems, including a light source of the medical imaging system, across a plurality of values of an operating parameter of the one or more light sources. The at least one memory may store instructions, where execution of the instructions by the one or more processors, may cause the computing system to perform operations. The operations may include receiving, at a first time: first image data from an imaging device of the medical imaging system captured during a medical procedure; and a first value of an operating parameter of the light source of the medical imaging system. The operations may also include: identifying, from the color shift data stored in the data store, first color shift data corresponding to the first value, where the first color shift data may include a measured change in pixel intensity values of one or more image components based on a first color shift of the light emitted by the light source when the operating parameter has the first value; determining one or more first compensation coefficients of a color correction function based on the first color shift data to compensate for the measured change in the pixel intensity values of the one or more image components; applying the color correction function, based on the one or more first compensation coefficients, to the first image data to generate first color corrected image data; and providing, to a display associated with the computing system, the first color corrected image data for display. The operations may further include receiving, at a second time different from the first time: second image data from the imaging device captured during the medical procedure; and a second value of the operating parameter of the light source, wherein the second value is different from the first value. The operations may yet further include: identifying, from the color shift data stored in the data store, second color shift data corresponding to the second value, where the second color shift data may indicate a measured change in pixel intensity values of one or more image components based on a second color shift of the light emitted by the light source when the operating parameter has the second value; determining one or more second compensation coefficients of the color correction function based on the second color shift data to compensate for the measured change in the pixel intensity values of the one or more image components; applying the color correction function, based on the one or more second compensation coefficients, to the second image data to compensate for the second color shift to generate second color corrected image data; and providing, to the display, the second color corrected image data for display.

In any of the exemplary computing systems disclosed herein, the operations may also include determining a difference between the second value and the first value is above a predefined threshold value. The operating parameter may be a current of the light source or a temperature of the light source.

A method for color correcting images performed by a computing system, may include receiving, at a first time: first image data from an imaging device of a medical imaging system captured during a medical procedure; and a first value of an operating parameter of a light source of the medical imaging system, where a color of light emitted from the light source may shift based on values of the operating parameter, and where the operating parameter may be a current of the light source or a temperature of the light source. The method may also include determining a first color shift based on the first value, applying a color correction function to the first image data to compensate for the first color shift to generate first color corrected image data, and providing, to a display associated with the computing system, the first color corrected image data for display. The method may further include receiving, at a second time different from the first time: second image data from the imaging device captured during the medical procedure; and a second value of the operating parameter of the light source, where the second value may be different from the first value. The method may yet further include determining a difference between the second value and the first value is above a predefined threshold value, and based on the difference being above the predefined threshold value: determining a second color shift based on the first value; applying the color correction function to the second image data to compensate for the second color shift to generate second color corrected image data; and providing, to the display, the second color corrected image data for display.

In any of the exemplary methods disclosed herein, the predefined threshold value may be based on a type of the operating parameter. A first predefined threshold value associated with the current of the light source may be less than a second predefined threshold value associated with the temperature of the light source.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an exemplary environment in which image color calibration and/or correction processes may be implemented.

FIG. 2 depicts an exemplary image color calibration method.

FIG. 3 depicts an exemplary image color correction method.

FIG. 4 depicts an exemplary system flow diagram for color correcting images.

FIG. 5 depicts an example of a computing device.

### DETAILED DESCRIPTION

As briefly discussed in the background, endoscopic imaging systems configured to capture images of a target site during a medical procedure include, among other devices, at least one light source and at least one imaging device. Some endoscopic imaging systems may drive the light source at a constant current throughout the medical procedure, and the imaging device gain and exposure time may be adjusted to achieve acceptable pixel saturation. To improve lighting control, other endoscopic imaging systems may adjust a light intensity of the light source first, and only adjust the imaging device gain and exposure time once the dynamic range of the light source has been exhausted. However, adjustment of the light intensity causes changes to an amount of current flowing through the light source, and when the current changes, a spectrum of the light source will vary. The variation in the spectrum results in a color shift affecting how image data of the target site is captured and reproduced by the endoscopic imaging system.

For example, the light source may be a light emitting diode (LED). A LED is a semiconductor device that emits light when current flows through it. Electrons in the semiconductor may recombine with electron holes and release energy in the form of photons, where a color of the light (corresponding to the energy of the photons) is determined by the energy required for electrons to cross a band gap of the semiconductor. White light may be obtained by using multiple semiconductors or a layer of light-emitting phosphor on the semiconductor. For example, typically, a white LED uses a blue LED photon pump allied to an yttrium aluminum garnet (YAG) phosphor. A first portion of the photons from the blue LED may pass through the phosphor unaffected, while a second portion of the photons may be absorbed by the phosphor material and emitted from a green portion of the spectrum through yellow, orange, and red portions of the spectrum. The human eye may perceive the combination as white light.

When a current of the LED is lower, the light emitted by the LED may be less blue (e.g., warmer). In contrast, when the current of the LED is higher, the phosphor becomes less efficient and the blue emission becomes more dominant, causing the light emitted by the LED to be increasingly blue (e.g., cooler). In other words, higher current values of the LED increase the percentage of higher energy photons (e.g., the higher energy photons having shorter wavelengths), which shift the spectrum of the emitted light towards the blue region. Accordingly, pixel value intensities of a blue component of image data of the target site may increase as the current of the LED increases.

Variations in other operating parameters of the light source, such as a temperature, can also cause variation in the spectrum of light emitted by the light source. For example, higher temperatures of a substrate of the LED change (e.g., lower) the bandgap of the semiconductor, which, in isolation, causes the photons emitted to shift toward the red region of the spectrum. However, because an increase in temperature may be resulting from an increase in the current of the LED, and the current has a more dominant effect over color than the temperature, the spectrum of the emitted light may overall still be shifted towards the blue region of the spectrum.

Aspects of this disclosure are directed to image color calibration and correction techniques that enable real-time image color correction during a medical procedure to compensate for light source spectrum variation and resulting color shift in captured image data based on one or more light source operating parameters. For example, during an end of line calibration process for a medical imaging system, a plurality of color shifts in light emitted by a light source of the medical imaging system may be characterized across a plurality of values of the one or more light source operating parameters to obtain color shift data specific to the light source (e.g., the characterization is performed per light source). As another example, the characterization may be performed across light sources of a plurality of medical imaging systems during a product development life cycle, for example, to obtain more generalized color shift data (e.g., averaged color shift data across the light sources).

The color shift data may indicate a measured change (e.g., an amount or percentage increase or decrease) in pixel intensity values of one or more components of image data (e.g., red, green, and/or blue components) when a given operating parameter of the light source is at a given value. The operating parameters may include a current of the light source, a temperature of the light source, and/or any other operating parameter of the light source that causes a shift in the color of the light emitted by the light source. The color shift data may be stored by and/or otherwise may be accessible by a computing system communicatively connectable to the medical imaging system. The color shift data may be used in image color correction processes performed by the computing system during a medical procedure.

To provide an illustrative image color correction process, image data from the imaging device and a first value of an operating parameter of the light source may be received. A color shift of light emitted from the light source may be determined based on the first value of the operating parameter, and a color correction function may be applied to the image data to compensate for the color shift to generate color corrected image data. For example, first color shift data corresponding to the first value may be identified using the stored color shift data. The first color shift data may indicate a measured change in pixel intensity values of one or more components of the image data based on the color shift of the light emitted by the light source when the operating parameter has the first value. One or more compensation coefficients of the color correction function may then be determined based on the first color shift data to compensate for the measured change in the pixel intensity values of the one or more components of the image data. Applying the color correction function based on the compensation coefficients may eliminate, or at least minimize, the color differences in the image data caused by the color shift. The color corrected image data may then be provided for display on one or more user interfaces, displays, and/or other external devices.

In some examples, first values of multiple operating parameters of the light source may be received (e.g., both a first value of current and a first value of temperature may be received). In such examples, the operating parameter having a dominant effect on the color shift (e.g., causing a larger measured change in the pixel intensity values of one or more image components) may be used to determine the color shift, for example, to apply the correction function accordingly.

The above image color correction process may be repeated one or more times throughout a duration of the medical procedure to enable for image color correction in real-time, for example, as values for the one or more operating parameters change. For example, as previously discussed, intensity of light emitted by the light source may be adjusted during the medical procedure for improved lighting control, causing current and/or temperature to change, which in turn affects the color of the light emitted by the light source. Therefore, based on new values received for one or more of the operating parameters (e.g., a second value, a third value, etc. that are different from the first value), the above process may be repeated to determine subsequent color shifts, and corresponding compensation coefficients of the color correction function to apply to the image data to generate the color corrected image data.

In some examples, a lag or delay in a timing of the color shift by the light source in response to a change in the operating parameter values may be accounted for when applying the color correction function to the image data. In further examples, to prevent a constant changing of the compensation coefficients of the color correction function over small changes in operating parameters, the process may be repeated when a difference between a new value and a previous value received is above a predefined threshold value. Such thresholding may help mitigate and/or eliminate a flickering or other changes in the color corrected image data that is provided for display. Additionally, such thresholding may help to conserve processing resources of the computing system. In further examples, if the values received for one or more of the operating parameters are not included in the stored color shift data, interpolation techniques may be applied using neighboring values.

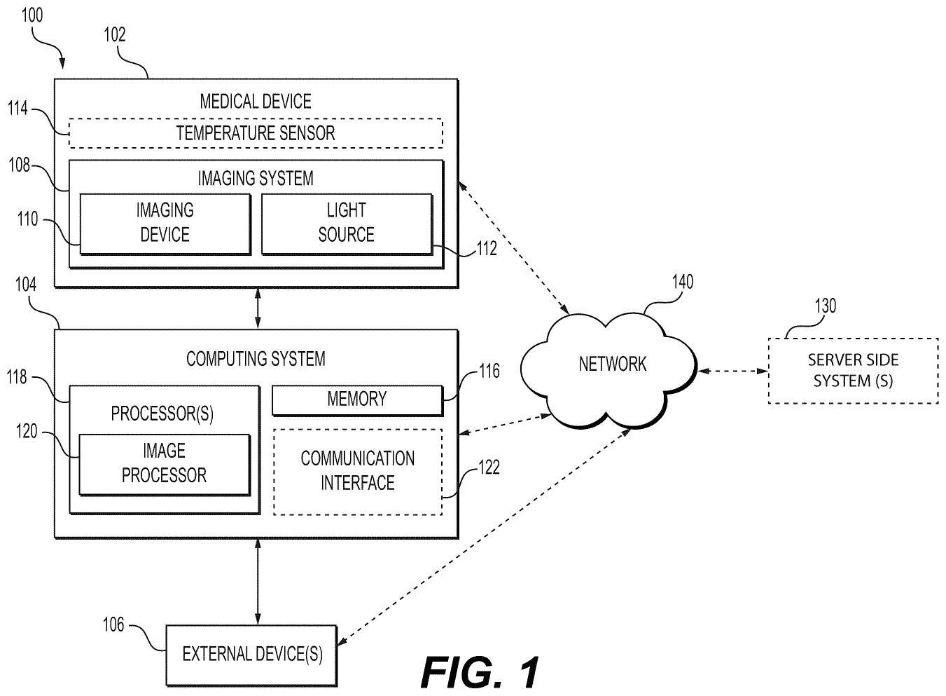

FIG. 1 depicts an exemplary environment 100 in which image color calibration and/or correction processes may be implemented. Environment 100 may include one or more of a medical device 102, a computing system 104, one or more external device(s) 106, one or more optional server side system(s) 130, and/or a network 140.

Medical device 102 may be used to perform a diagnostic and/or interventional medical procedure on a patient, hereinafter referred to as medical procedure for brevity. Medical device 102 may be an endoscope or other type of scope, such as a bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples.

Medical device 102 may include an imaging system 108. Imaging system 108 may include at least one imaging device 110 and at least one light source 112. Imaging device 110 and/or light source 112 may be located at a distal end of medical device 102 (e.g., at a distal tip of medical device 102). Imaging device 110 may be configured to capture image signals as the distal end of medical device 102 is inserted into and navigated through a body lumen of the patient to a target site during the medical procedure. Imaging device 110 may include one or more cameras, one or more image sensors, endoscopic viewing elements, or optical assemblies including one or more image sensors and one or more lenses, among other similar devices. Light source 112 may be configured to illuminate areas of the patient's body (e.g., the target site) during the medical procedure to facilitate imaging of the target site by imaging device 110. Light source 112 may include one or more LEDs, incandescent light sources, optical fibers, and/or other illuminators.

In some examples, to improve lighting control, an intensity of light emitted by light source 112 may be adjusted throughout the medical procedure. Adjustment of the light intensity causes a change in an amount of current flowing through light source 112, and when the current is changed, a spectrum of the light emitted by light source 112 may vary. The variation in the spectrum may result in a color shift affecting how the features of the target site are captured by imaging device 110. Throughout the medical procedure, light source 112 may be configured to detect and/or measure the amount of the current flowing through light source 112 for conversion into an electrical signal (e.g., a current signal) to indicate a current value. The current value may be provided to computing system 104 for use in one or more image color correction processes to compensate for the color shift.

Variations in other operating parameters of light source 112, such as a temperature, individually or in conjunction with the current, can also cause variation in the spectrum of the light emitted by light source 112, resulting in the color shift. Therefore, medical device 102 may also include an optional temperature sensor 114. Optional temperature sensor 114 may be positioned adjacent to light source 112. Optional temperature sensor 114 may be configured to measure a temperature of light source 112. For example, optional temperature sensor 114 may detect and/or measure coldness and/or heat for conversion into an electrical signal (e.g., a temperature signal) to indicate a temperature value. In examples where light source 112 includes one or more LEDs, optional temperature sensor 114 may be configured to measure the temperature of a substrate of the one or more LEDs. The temperature value may be provided to computing system 104 for use in one or more image color correction processes to compensate for the color shift.

One or more components of medical device 102, including imaging system 108 and the components thereof, may be communicatively connectable to computing system 104 via wired connections and/or wireless connections (e.g., over network 140) to enable communication of various signals between medical device 102 and computing system 104. For example, image signals captured by imaging device 110 may be received by computing system 104. Additionally, current signals from light source 112 and/or temperature signals from optional temperature sensor 114 may be received by computing system 104. In some examples, computing system 104 may provide lighting signals to medical device 102 to cause light source 112 to emit light and/or emit light at a given intensity.

In some examples, computing system 104 is a computing device, controller, or other similar standalone processing unit separate from medical device 102. In other examples, computing system 104 may be integrated with medical device 102. For example, computing system 104 may be positioned in a handle of medical device 102. In other examples, computing system 104 may be positioned at the distal end of medical device 102.

Computing system 104 may include a memory 116 and one or more processor(s) 118. Memory 116 may store instructions to be executed by processor(s) 118 to cause computing system 104 to perform corresponding operations. At least a portion of the instructions stored in memory 116 may include an image color correction process. Memory 116 may also include one or more data stores. Additionally or alternatively, computing system 104 may also include one or more data stores separate from memory 116. Processor(s) 118 may include at least one image processor 120. Image processor 120 may be configured to process the image signals captured by imaging device 110 and received by computing system 104 to generate image data. Additionally, image processor 120 may be configured to perform the image color correction process to generate color corrected image data. As described in greater detail below, the color corrected image data may be generated based on the image signals and one or more other signals associated with one or more operating parameters of light source 112 (e.g., current signals and/or temperature signals) to compensate for a color shift resulting from a corresponding state of the one or more operating parameters, as described in detail below. In some examples, image processor 120 may be a field-programmable gate array (FPGA), a digital signal processing (DSP) processor, a graphics processing unit (GPU), or the like. Dependent on a type of the image processor (e.g. basic or more advanced), image processor 120 may be capable of performing a variety of additional image processing operations, including more complex artificial intelligence (AI) or machine learning-based techniques.

Computing system 104 may further include an optional communication interface 122 for providing connectivity to network 140. Optional communication interface 122 may also provide connectivity to medical device 102 and/or external device(s) 106. In some examples, a communicative connection between computing system 104 and medical device 102 (or components thereof) and/or computing system 104 and external device(s) 106 may be at least partially supported via network 140.

At least one of external device(s) 106 may be a display device configured to display image data, such as the color corrected image data generated by computing system 104. The display device may be a monitor, computing device screen, touch screen display device, etc. In some examples, the display device may be a separate device from computing system 104 that is connectable to computing system 104 via wired and/or wireless connections. In other examples, the display device may be a display of computing system 104 itself.

In some examples, computing system 104 may generate, or may cause to be generated, one or more graphical user interfaces based on instructions or information stored in the memory 116, instructions or information received from one or more of the optional server side system(s) 130, and/or the like and may cause the graphical user interfaces to be displayed via the display device. The graphical user interfaces may be, e.g., application interfaces or browser user interfaces and may include text, selection controls, and/or the like, in addition to the displayed color corrected image data. The display device may include a touch screen or a display with other input systems (e.g., a mouse, keyboard, voice, etc.) for an operator of computing system 104 to control functions of one or more of computing system 104, medical device 102 (or components thereof) via computing system 104, and/or the display device. As one example, the operator may select one or more of the control elements displayed on a graphical user interface of the display device to adjust an intensity of the light emitted by light source 112. The selection may be received by computing system 104 and cause corresponding lighting signals to be transmitted from computing system 104 to light source 112.

In some examples, external device(s) 106 may further include one or more third party processing systems, such as AI processing systems, connectable to computing system 104. Exemplary AI processing systems may be configured to receive the image data and/or the color corrected image data generated by computing system 104 as an input, and may also be configured to process the image data (and optionally other input data) to generate augmented image data.

One or more components of environment 100, such as medical device 102, computing system 104, and/or external device(s) 106, may be capable of network connectivity, and may communicate with one another over a wired or wireless network, such as network 140. Network 140 may be an electronic network. Network 140 may include one or more wired and/or wireless networks, such as a wide area network (“WAN”), a local area network (“LAN”), personal area network (“PAN”), a cellular network (e.g., a 3G network, a 4G network, a 5G network, etc.), or the like. In other examples, the components of environment 100 may communicate and/or connect to network 140 over universal serial bus (USB) or other similar local, low latency connections or direct wireless protocol.

In some embodiments, network 140 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. Components of environment 100 may be connected via network 140, using one or more standard communication protocols, such that the component may transmit and receive communications from each other across network 140.

In some examples, when one or more of the components of environment 100 are capable of connecting to network 140, environment 100 may also include one or more optional server side system(s) 130. Optional server side system(s) 130 may include one or more of remote image processing systems configured to perform at least a portion of the image processing (e.g., to conserve local resources of computing system 104 when network connectivity is available). Additionally or alternatively, server side system(s) 130 may include data storage systems for storing the image data and/or the color corrected image data generated by computing system 104, and/or augmented image data generated by third party processing systems (e.g., one or more of external device(s) 106). In some examples, at least one of the data storage systems may include a picture archiving and communication system (PACS) that stores the image data, color corrected image data, and/or augmented image data, along with other types of imaging data from various imaging modalities (e.g., ultrasound, magnetic resonance, nuclear medicine imaging, positron emission tomography, computed tomography, mammograms, digital radiography, histopathology, etc.). Further, optional server side system(s) 130 may include endoscopic report writer systems configured to facilitate generation of a report based on the image data, color corrected image data, and/or augmented data.

Although various components in environment 100 are depicted as separate components in FIG. 1, it should be understood that a component or portion of a component in environment 100 may, in some embodiments, be integrated with or incorporated into one or more other components. In some embodiments, operations or aspects of one or more of the components discussed above may be distributed amongst one or more other components. Any suitable arrangement and/or integration of the various systems and devices of environment 100 may be used.

The specific examples included throughout the present disclosure implement endoscopic imaging systems configured to perform image color correction processes in real-time during a medical procedure based on LED operating parameters affecting color shift, such as current and/or temperature. However, it should be understood that techniques according to this disclosure may be adapted to other medical imaging systems having varying types of light sources, and therefore, the image color correction processes may further be based on different and/or additional types of light source operating parameters that affect color shift (e.g., dependent on light source type), such as lifetime for incandescent light sources. It should also be understood that the examples above are illustrative only. The techniques and technologies of this disclosure may be adapted to any suitable activity.

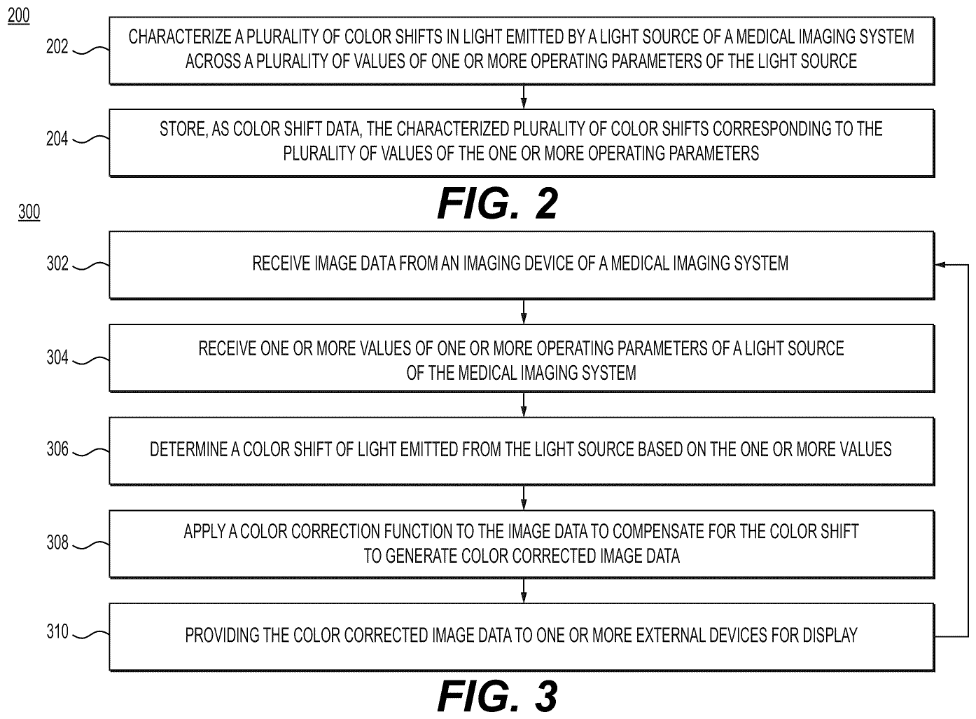

FIG. 2 depicts an exemplary image color calibration method 200, hereinafter method 200, for determining color shift data. In some examples, one or more steps of method 200 may be performed by computing system 104. In other examples, one or more steps of method 200 may be performed by another computing device or system, and the color shift data determined may be provided to computing system 104 for storage.

At step 202, a plurality of color shifts in light emitted by a light source of a medical imaging system may be characterized across a plurality of values of one or more operating parameters of the light source. In one example, the light source being characterized may be light source 112 of medical device 102 during an end of line calibration process, such that the plurality of color shifts are specific to light source 112. That is, the characterization is performed per medical device 102. In another example, the color shifts may be characterized more generally during a product development life cycle of a plurality of medical devices 102. That is, the characterization may be performed across a plurality of lights sources 112 of a plurality of medical devices 102.

Example operating parameters may include a current, a temperature, and/or any other operating parameters that affect color shift based on a type of light source 112 (e.g., lifespan if light source 112 is an incandescent light source). For each operating parameter, parameter-specific color shift data may be determined that includes a range of operating parameter values and a corresponding color shift measured at each value over the range of operating parameter values. The color shift measured at a given value may indicate a measured change (e.g., an amount or percentage increase or decrease) in pixel intensity values of one or more components of image data captured by imaging device 110, such as red, green, and/or blue components, when the operating parameter of light source 112 is at the given value. A timing associated with the color shift may also be characterized and stored in association with the measured change at the given value. The timing may include a measured time period from a detection of the operating parameter of light source 112 at the first value to an observation of the color shift. As one example, the timing may include a particular time period (e.g., a particular number of microseconds) from when the current is detected at a given value until when the measured change is observed. As another example, the timing may include a particular time period (e.g., particular number of seconds) from when the temperature is detected a given value until when the measured change is observed. In some examples, a calibration mapping may be generated from the parameter-specific color shift data for each operating parameter.

As one example, current flowing through light source 112 may be adjusted over a range of current values by, for example, adjusting an intensity of light emitted by light source 112 (e.g., by sending signal(s) from computing system 104 to light source 112 to emit light at given intensities). Color shifts, including associated timings of the color shifts, may be determined at the respective current values over the range of operating parameter values based on image data captured by imaging device 110 when light source 112 is operating at the respective current values. The determined color shifts may be stored as color shift data associated with current (e.g., current-specific color shift data). For example, the current-specific color shift data may indicate that, given a current of x milliamps flowing through light source 112, pixel intensity values of a first component of image data captured by imaging device 110 are going to be increased by y %. The first component may be a primary component affected (e.g., the spectrum ultimately shifts toward the first component). However, light source 112, when operating at a current of x milliamps, may also at least minimally affect remaining components of the image data. For example, if the first component is the blue component of the image data, the red and/or green components of the image data may also be affected. Therefore, the current-specific color shift data may also indicate the effects on pixel intensity values of the remaining components. A current calibration mapping may be generated based on the current-specific color shift data.

As another example, a temperature of light source 112 may be adjusted over a range of temperature values. Color shifts, including associated timings of the color shifts, may be determined at the respective temperature values over the range of temperature values based on image data captured by imaging device 110 at the respective temperature values. The determined color shifts may be stored as color shift data associated with temperature (e.g., temperature-specific color shift data). For example, the temperature-specific color shift data may indicate that, given light source 112 having a temperature of a degrees, pixel intensity values of a first component of image data captured by imaging device 110 are going to be increased by β %. The first component may be the primary component affected (e.g., the spectrum ultimately shifts toward the first component). However, light source 112, when operating at a temperature of a degrees may also at least minimally affect remaining components of the image data. Therefore, the temperature-specific color shift data may also indicate the effects on pixel intensity values of the remaining components. A temperature calibration mapping may be generated based on the temperature-specific color shift data. In some examples, the temperature values may be measured via a sensor, such as, for example, optional temperature sensor 114, positioned adjacent to light source 112. In other examples, the temperature values may be inferred based on a value of a current of light source 112, for example, over time, and one or more other known values associated with light source, such as a known ambient temperature and a known junction thermal resistance of light source 112.

In some examples, a color shift and an associated timing of the color shift may be determined for each potential or allowed value of an operating parameter, such as each allowed current value and/or allowed temperature value based on specifications of light source 112. In other examples, the color shift may be determined for a subset of allowed values of the operating parameter, and interpolation techniques may be applied to obtain color shift data for values in between the subset of allowed values.

At step 204, the characterized plurality of color shifts corresponding to the plurality of values of the one or more operating parameters may be stored as the color shift data (e.g., the current-specific color shift data, temperature-specific color shift data, etc. may be stored). When mappings are generated from the color shift data, the mappings may be stored. In some examples, the color shift data may be stored in the one or more data stores of memory 116 and/or other local data storage of computing system 104 (e.g., within one or more data stores of computing system 104 separate from memory 116). Additionally or alternatively, the color shift data may be stored in data storage systems associated with the optional server side system(s) 130.

As the spectrum of light emitted by light source 112 shifts due to changes in one or more of the operating parameters of light source 112 during a medical procedure, different compensation coefficients of a color correction function may be appropriate to generate an image with minimized color differences (e.g., to generate a color corrected image that eliminates and/or at least mitigates the color shift resulting from the spectrum shift). Therefore, as described in detail below, the stored color shift data may be subsequently retrieved to determine one or more compensation coefficients of the color correction function for application in real-time to image data captured by imaging device 110 during the medical procedure in order to compensate for color shift based on one or more current operating parameters of light source 112.

Accordingly, certain aspects may be performed for determining color shift data. Method 200 described above is provided merely as an example, and may include additional, fewer, different, or differently arranged steps than depicted in FIG. 2.

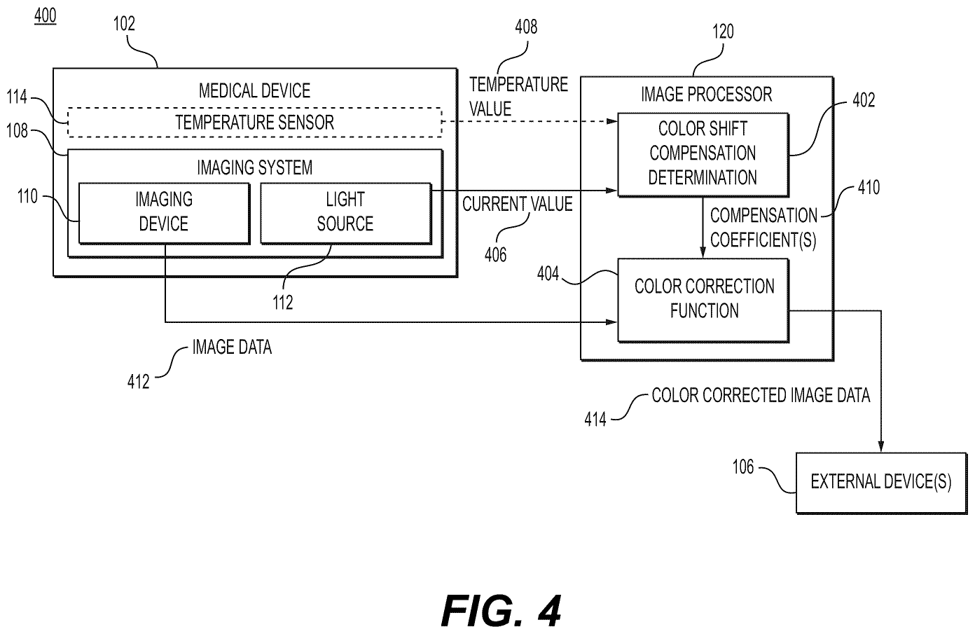

FIG. 3 depicts an exemplary image color correction method 300, hereinafter method 300. FIG. 4 depicts an exemplary system flow diagram 400 for color correcting images by performing one more steps of method 300 of FIG. 3, for example. Referring concurrently to FIGS. 3 and 4, in some examples, one or more steps of method 300 may be performed by computing system 104, and more specifically image processor 120 of computing system 104. The method 300 may be performed during a medical procedure utilizing medical device 102. For example, as medical device 102 is inserted into and navigated through a body lumen of the patient to a target site during the medical procedure, imaging device 110 may be capturing images of areas of the patient's body as light source 112 is illuminating the areas of the patient's body (e.g., the target site). Method 300 described below is provided merely as an example, and may include additional, fewer, different, or differently arranged steps than depicted in FIG. 3.

At step 302, image data 412 may be received from imaging device 110 of a medical imaging system, such as imaging system 108 of medical device 102. As mentioned above, image data 412 may be received (e.g., from imaging system 108) during the insertion and/or navigation of medical device 102 to the target site.

At step 304, one or more values of one or more operating parameters of light source 112 of the medical imaging system may be received. For example, a current value 406 of a current of light source 112 may be received from light source 112 (e.g., received as a current signal from light source 112). Additionally or alternatively, a temperature value 408 may be received from optional temperature sensor 114 (e.g., received as a temperature signal from light source 112). In other examples, such as when medical device 102 does not include optional temperature sensor 114, temperature value 408 may be inferred based on a plurality of current values over time, including current value 406, and one or more other known values associated with light source 112. For example, image processor 120 may determine temperature value 408 based on a known ambient temperature, a known junction thermal resistance of light source 112, and current value 406 over time. Other example operating parameters may include any other operating parameter of light source 112 that affect color shift, which may be dependent on a type of light source 112. For example, if light source 112 is an incandescent light, a lifespan of light source 112 may be received and/or determined for use in step 306.

At step 306, a color shift of light emitted from light source 112 may be determined based on the one or more values received at step 304 (e.g., via a color shift compensation determination process 402). For example, image processor 120 may receive the color shift data that was determined and stored locally in the one or more data stores of computing system 104 (e.g., in data stores of memory 116 and/or data stores separate from memory 116) and/or stored remotely in a data storage system of the optional server side system(s) 130, for example, as described with reference to method 200 of FIG. 2. In some examples, the color shift data received may be a calibration mapping. The color shift data may indicate how pixel intensity values for each of one or more components (e.g., each of red, green, and/or blue components) of the image data captured by imaging device 110 are affected when the one or more operating parameters are detected as being at the one or more values received at step 304. The color shift data may also indicate an associated timing of the effects on the pixel intensity values for each of one or more components following the detection of the one or more operating parameters as being at the one or more values.

Image processor 120 may use the color shift data and/or the calibration mapping for a given operating parameter to identify a color shift of light (e.g., a measured change in pixel intensity values of one or more components of image data) corresponding to the value of the given operating parameter received at step 304. For example, if current value 406 of x milliamps is received, a y₁ change in the red component of image data 412, a y₂ change in the green component of image data 412, and/or a y₃ change in the blue component of image data 412 corresponding to x milliamps may be identified within the current-specific color shift data and/or current calibration mapping. Additionally, an associated timing from a detection of light source 112 operating at x milliamps to an observation of the changes may be identified. As another example, if temperature value 408 of a degrees is received, a β₁ change in the red component of image data 412, a β₂ change in the green component of image data 412, and/or a β₃ change in the blue component of image data 412 corresponding to a degrees may be identified within the temperature-specific color shift data and/or temperature calibration mapping. Additionally, an associated timing from a detection of light source 112 operating at a degrees to an observation of the changes may be identified. In some examples, if the value of the given parameter is not included in the color shift data and/or calibration mapping, interpolation techniques may be applied using neighboring values to identify the corresponding change in one or more image components.

Additionally, in further examples, when multiple operating parameters may affect the color shift (e.g., both current and temperature) at the respective given values, the operating parameter having a dominant effect may be used to characterize the color shift. The operating parameter having the dominant effect may be the operating parameter that causes a largest measured change (e.g., a largest amount or percentage increase or decrease) in pixel value intensities of an image component.

Further, as part of color shift compensation determination process 402, the determined color shift may then be used to determine one or more coefficients of a color correction function 404 to compensate for the color shift (e.g., compensation coefficient(s) 410). In some examples, color correction function 404 may be a 3×3 color correction matrix that is applied to image data 412 to achieve color corrected image data 414 on a per pixel basis to minimize color differences resulting from (e.g., mitigate or eliminate effects or) the color shift. As previously discussed with reference to FIG. 2, one of the red, green, and/or blue components of image data 412 may be the primary component affected by the color shift (e.g., the spectrum will shift toward the primary component). However, remaining components may also at least be minimally affected. Therefore, compensation coefficients 410 may also be determined for off-diagonal elements of the color correction matrix (e.g., elements corresponding to the remaining components). For example, the 3×3 color correction matrix may be comprised of three rows. Each row may include three determined compensation coefficients 410 to multiply against red, green, and blue pixel value intensities, respectively, that are detected in one of the red, green, or blue color channels by imaging device 110.

The determined compensation coefficient(s) 410 may result in an inverse of the identified change in the pixel intensity values of the one or more image components. For example, if based on current value 406, the color shift is a y₁ change in the red component of image data 412, a y₂ change in the green component of image data 412, and/or a y₃ change in the blue component of image data 412, compensation coefficient(s) 410 to counteract the respective y₁, y₂, and/or y₃ changes in the red, green, and blue components may be determined to minimize color differences caused by the color shift. As another example, if based on temperature value 408, the color shift is a β₁ change in the red component of image data 412, a β₂ change in the green component of image data 412, and/or a β₃ change in the blue component of image data 412, compensation coefficient(s) 410 to counteract the respective β₁, β2, and/or β₃ changes in the red, green, and blue components may be determined to minimize color differences caused by the color shift. Determined compensation coefficient(s) 410 may be provided for use in color correction function 404.

At step 308, color correction function 404 may be applied to image data 412 to compensate for the color shift to generate color corrected image data 414. For example, color correction function 404 may include compensation coefficient(s) 410 to compensate for the color shift. A timing at which the color correction function 404 may be applied may be based on the associated timing of the color shift indicated by the color shift data and/or the calibration mapping.

At step 310, color corrected image data 414 may be provided to one or more of external device(s) 106 for display. For example, external device(s) 106 may include a display device communicatively coupled to and/or a display device integrated with computing system 104. Display of color corrected image data 414 may help an operator of medical device 102 visually navigate to and more clearly identify features within the target site associated with the medical procedure.

In other examples, color corrected image data 414 may be provided to one or more other external device(s) 106, such as third party, AI processing systems, connectable to computing system 104. Exemplary AI processing systems may be configured to receive color corrected image data 414 generated by computing system 104 as an input and may also be configured to process color corrected image data 414 to generate augmented image data. The augmented image data may then be provided for display via one or more of the above-described display devices.

Steps 302-310 of method 300 may be repeated throughout the medical procedure periodically and/or as changes in the one or more operating parameters of light source 112 occur in real or near-real time to enable “on the fly” color correction to image data received from imaging device 110 to account for the changes. For example, image data 412 received at step 302 may be first image data received from imaging device 110 at a first time, and the one or more values received at step 304 may be first values of the operating parameter(s) at the first time that are used to determine a first color shift. Color correction function 404 compensating for the first color shift may continue to be applied to first image data as it is received from imaging device 110 until a change in the operating parameter(s) is detected (e.g., at a second time different from the first time).

For example, at a second time during the medical procedure, second image data may be received from imaging device 110 at step 302, and one or more second values that are different from the one or more first values of the one or more operating parameters of light source 112 may be received at step 304. Accordingly, a second color shift of light emitted from light source 112 may be determined based on the one or more second values at step 306, and color correction function 404 may be applied to the second image data to compensate for the second color shift to generate second color corrected image data at step 308. For example, one or more different (e.g., second) compensation coefficient(s) 410 of color correction function 404 may be determined via color shift compensation determination process 402 to compensate for the second color shift. The second color collected image data may then be provided to one or more external devices for display at step 310. Color correction function 404 compensating for the second color shift may continue to be applied to the second image data as the second image data is received from imaging device 110 until a next change in the operating parameter(s) is detected (e.g., at a third time during the medical procedure).

When applying method 300 in real-time during the medical procedure, image processor 120 may apply hysteresis. For example, a lag or delay in the timing of the color shift in response to the change in the operating parameter values may be accounted for. For example, given an increase in temperature to a degrees, a lag or delay of z milliseconds may be expected prior to the shift in the color emitted by light source 112 to occur. Therefore, the color correction function with compensation coefficients determined to compensate for the color shift when the temperature is a degrees may be applied to image data that is being received z milliseconds later.

Additionally, as values of the operating parameters change, one or more predefined threshold values may be applied before changing color correction function 404 (e.g., before changing compensation coefficient(s) 410 of color correction function 404). For example, each predefined threshold value may represent a minimum change (in absolute value) for a given operating parameter that is to be exceeded prior to changing compensation coefficient(s) 410 in color correction function 404. In other words, an absolute value difference of a current value and a preceding value (e.g., a first value and a second value) may be determined to be above a predefined threshold value before changing color correction function 404.

Each predefined threshold value may be based on a type of the operating parameter and/or a color shift sensitivity to changes in operating parameter values of that type. For example, a first predefined threshold value for current may be a lower value (e.g., allows a lower minimum change) than a second predefined threshold value for temperature. This thresholding may help to prevent a constant changing of color correction function 404 over small changes in operating parameters (e.g., prevents image processor 120 from being overreactive). Such thresholding may help mitigate and/or eliminate a flickering in color corrected image data 414 that is provided to external device(s) 106 for display. Additionally, such thresholding may help to conserve processing resources of computing system 104 by reducing a number of iterations or instances that at least steps 306 and 308 are performed, for example.

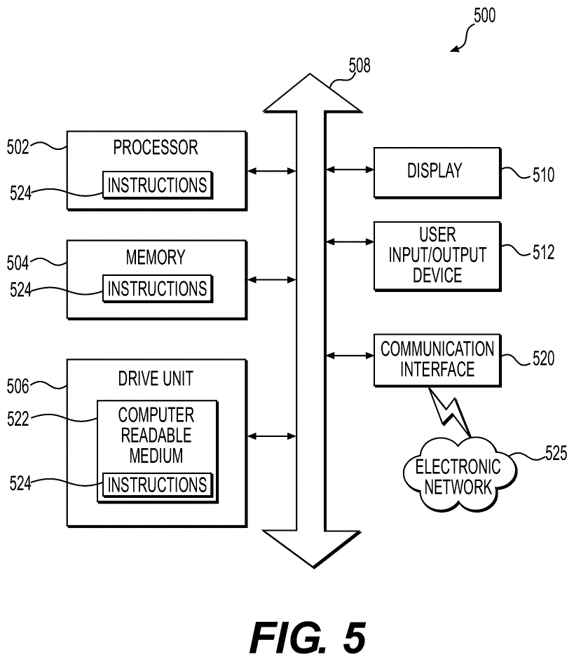

FIG. 5 depicts an example of a computer 500. FIG. 5 is a simplified functional block diagram of computer 500 that may be configured as a device for executing processes, steps, or operations depicted in, or described with respect to, FIGS. 1-4 and, according to exemplary embodiments of the present disclosure. For example, computer 500 may be configured as one or more of medical device 102, computing system 104, external device(s) 106, server side system(s) 130, and/or another device or component according to exemplary embodiments of this disclosure. In various embodiments, any of the systems herein may be or include computer 500 including, e.g., a data communication interface 520 for packet data communication. Computer 500 may communicate with one or more other computers, for example, using an electronic network 525 (e.g., via data communication interface 520). Electronic network 525 may include a wired or wireless network, for example, similar to network 140 depicted in FIG. 1.

Computer 500 also may include a central processing unit (“CPU”), in the form of one or more processors 502, for executing program instructions 524. Program instructions 524 may include at least instructions for performing image processing, including image color correction (e.g., if computer 500 is computing system 104).

Computer 500 may include an internal communication bus 508. Computer 500 may also include a drive unit 506 (such as read-only memory (ROM), hard disk drive (HDD), solid-state disk drive (SDD), etc.) that may store data on a computer readable medium 522 (e.g., a non-transitory computer readable medium), although computer 500 may receive programming and data via network communications. Computer 500 may also have a memory 504 (such as random-access memory (RAM)) storing instructions 524 for executing techniques presented herein. It is noted, however, that in some aspects, instructions 524 may be stored temporarily or permanently within other modules of computer 500 (e.g., processor 502 and/or computer readable medium 522). Computer 500 also may include user input and output devices 512 and/or a display 510 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A method for color correcting images performed by a computing system, the method comprising:
receiving, at a first time:
first image data from an imaging device of a medical imaging system captured during a medical procedure; and
a first value of an operating parameter of a light source of the medical imaging system, wherein a color of light emitted from the light source shifts based on values of the operating parameter;
determining a first color shift based on the first value;
applying a color correction function to the first image data to compensate for the first color shift to generate first color corrected image data;
providing, to a display associated with the computing system, the first color corrected image data for display;
receiving, at a second time different from the first time:
second image data from the imaging device captured during the medical procedure; and
a second value of the operating parameter of the light source, wherein the second value is different from the first value;
determining a second color shift based on the second value;
applying the color correction function to the second image data to compensate for the second color shift to generate second color corrected image data; and
providing, to the display, the second color corrected image data for display.

2. The method of claim 1, wherein a plurality of color shifts in light emitted by one or more light sources of one or more medical imaging systems, including at least the light source of the medical imaging system, are characterized across a plurality of values of the operating parameter to obtain and store color shift data.

3. The method of claim 2, wherein determining the first color shift comprises:
identifying first color shift data corresponding to the first value within the stored color shift data, the first color shift data indicating a measured change in pixel intensity values of one or more image data components based on the first color shift of the light emitted by the light source when the operating parameter has the first value.

4. The method of claim 3, wherein applying the color correction function to the first image data comprises:
determining one or more first compensation coefficients of the color correction function based on the first color shift data to compensate for the measured change in the pixel intensity values of the one or more image data components.

5. The method of claim 3, wherein the first color shift data further indicates a timing associated with the measured change in the pixel intensity values of one or more image data components when the operating parameter has the first value, and applying the color correction function to the first image data comprises:
applying the color correction function to the first image data based on the timing.

6. The method of claim 2, wherein determining the second color shift comprises:
identifying second color shift data corresponding to the second value within the stored color shift data, the second color shift data indicating a measured change in pixel intensity values of one or more image data components based on the second color shift of the light emitted by the light source when the operating parameter has the second value.

7. The method of claim 6, wherein applying the color correction function to the second image data comprises:
determining one or more second compensation coefficients of the color correction function based on the second color shift data to compensate for the measured change in the pixel intensity values of the one or more image data components.

8. The method of claim 6, wherein the second color shift data further indicates a timing associated with the measured change in the pixel intensity values of one or more image data components when the operating parameter has the second value, and applying the color correction function to the second image data comprises:
applying the color correction function to the second image data based on the timing.

9. The method of claim 1, further comprising:
determining a difference between the second value and the first value is above a predefined threshold value.

10. The method of claim 1, wherein the operating parameter is a current of the light source, and receiving the first value and the second value of the operating parameter comprises:
receiving a first value of the current and a second value of the current from the light source at the first time and the second time, respectively.

11. The method of claim 1, wherein the operating parameter is a temperature of the light source, and receiving the first value and the second value of the operating parameter comprises:
receiving a first value of the temperature and a second value of the temperature from a temperature sensor positioned adjacent to the light source at the first time and the second time, respectively.

12. The method of claim 1, wherein the operating parameter is a temperature of the light source, and receiving the first value and the second value of the operating parameter further comprises:
inferring a first value of the temperature and a second value of the temperature based on a known ambient temperature, a known junction thermal resistance of the light source, and a value of a current of the light source received at the first time and the second time, respectively.

13. The method of claim 1, wherein applying the color correction function to the first image data comprises:
determining a first timing associated with the first color shift, the first timing including a measured time period from a detection of the operating parameter of the light source operating at the first value to an observation of the first color shift; and
applying the color correction function to the first image data based on the first timing.

14. The method of claim 1, wherein applying the color correction function to the second image data comprises:
determining a second timing associated with the second color shift, the second timing including a measured time period from a detection of the operating parameter of the light source operating at the second value to an observation of the second color shift; and
applying the color correction function to the second image data based on the second timing.

15. The method of claim 1, wherein the operating parameter is a first operating parameter, and the method further comprises:
receiving, at the first time, a first value of a second operating parameter of the light source in addition to the first value of the first operating parameter; and
determining the first operating parameter at the first value has a more dominant effect on the color of the light emitted from the light source than the second operating parameter at the first value, the determination causing the first color shift and an associated timing of the first color shift to be determined based on the first value of the first operating parameter.

16. A computing system for color correcting images that is communicatively connectable to a medical imaging system, the computing system comprising:
a data store storing color shift data obtained from a characterization of a plurality of color shifts in light emitted by one or more light sources of one or more medical imaging systems, including a light source of the medical imaging system, across a plurality of values of an operating parameter of the one or more light sources;
at least one memory storing instructions; and
one or more processors, including an image processor, wherein execution of the instructions by the one or more processors, causes the computing system to perform operations, including:
receiving, at a first time:
first image data from an imaging device of the medical imaging system captured during a medical procedure; and
a first value of an operating parameter of the light source of the medical imaging system;
identifying, from the color shift data stored in the data store, first color shift data corresponding to the first value, the first color shift data indicating a measured change in pixel intensity values of one or more image components based on a first color shift of the light emitted by the light source when the operating parameter has the first value;
determining one or more first compensation coefficients of a color correction function based on the first color shift data to compensate for the measured change in the pixel intensity values of the one or more image components;
applying the color correction function, based on the one or more first compensation coefficients, to the first image data to generate first color corrected image data;
providing, to a display associated with the computing system, the first color corrected image data for display;
receiving, at a second time different from the first time:
second image data from the imaging device captured during the medical procedure; and
a second value of the operating parameter of the light source, wherein the second value is different from the first value;
identifying, from the color shift data stored in the data store, second color shift data corresponding to the second value, the second color shift data indicating a measured change in pixel intensity values of one or more image components based on a second color shift of the light emitted by the light source when the operating parameter has the second value;
determining one or more second compensation coefficients of the color correction function based on the second color shift data to compensate for the measured change in the pixel intensity values of the one or more image components;
applying the color correction function, based on the one or more second compensation coefficients, to the second image data to compensate for the second color shift to generate second color corrected image data; and
providing, to the display, the second color corrected image data for display.

17. The computing system of claim 16, the operations further comprising:
determining a difference between the second value and the first value is above a predefined threshold value.

18. The computing system of claim 16, wherein the operating parameter is a current of the light source or a temperature of the light source.

19. A method for color correcting images performed by a computing system, the method comprising:
receiving, at a first time:
first image data from an imaging device of a medical imaging system captured during a medical procedure; and
a first value of an operating parameter of a light source of the medical imaging system, wherein a color of light emitted from the light source shifts based on values of the operating parameter, and wherein the operating parameter is one of a current of the light source or a temperature of the light source;
determining a first color shift based on the first value;
applying a color correction function to the first image data to compensate for the first color shift to generate first color corrected image data;
providing, to a display associated with the computing system, the first color corrected image data for display;
receiving, at a second time different from the first time:
second image data from the imaging device captured during the medical procedure; and
a second value of the operating parameter of the light source, wherein the second value is different from the first value;
determining a difference between the second value and the first value is above a predefined threshold value; and
based on the difference being above the predefined threshold value:
determining a second color shift based on the first value;
applying the color correction function to the second image data to compensate for the second color shift to generate second color corrected image data; and
providing, to the display, the second color corrected image data for display.

20. The method of claim 19, wherein the predefined threshold value is based on a type of the operating parameter, and wherein a first predefined threshold value associated with the current of the light source is less than a second predefined threshold value associated with the temperature of the light source.
